(19) United States
(12) Patent Application Publication
(10) Pub. No.: US 2012/0318259 A1
(43) Pub. Date: Dec. 20, 2012

(54) METHOD AND APPARATUS FOR PRODUCING FINE CONCENTRATED AEROSOL

(75) Inventor: Akper Sadykhov, Kiryat Mozkin (IL)

(73) Assignee: OMEGA LIFE SCIENCE LTD., Migdal Haemek (IL)

(21) Appl. No.: 13/521,718

(22) PCT Filed: Jan. 12, 2011
(86) PCT No.: PCT/IL2011/000038

§ 371 Date: Jul. 11, 2012

Related U.S. Application Data
(60) Provisional application No. 61/294,161, filed on Jan. 12, 2010.

(51) Int. Cl. A61M 11/00

(52) U.S. Cl. 128/200.22

(57) ABSTRACT

The invention relates to a method and apparatus for producing fine and concentrated aerosol from liquids, for various applications, by using rigid porous material, storage and easy streaming of the aerosol, (i.e. for inhalation), fast replacement of liquid carrier device and accuracy of output sprayed dose.

### FIELD OF INVENTION

The present invention relates to a method and apparatus enabling production of ultra fine and concentrated aerosol from liquids, for various applications, by using rigid porous material, enabling storage and easy streaming of the aerosol, (i.e. for inhalation), fast replacement of liquid carrier device and accuracy of output sprayed dose.

### BACKGROUND OF INVENTION

The efficiency and effectiveness of nebulizer technology depends on its ability to produce droplets of aerosol characterized by required parameters (i.e.: size, concentration etc.) since these parameters have a direct impact on the performance of the aerosolized material (i.e. therapeutic effect). Additional needs are to eliminate product loss during operation and to allow convenience of use.

From existing professional bibliography it is known that: Effective lung healing treatment requires a certain dose of medicine composed of particles sized 0.5 to 2 micrometer.

### PRIOR ART

### U.S Patents

| Pat. No. | | Date of Patent |
|---|---|---|
| 4,743,407 | Externally pressurized porous | May 10, 1988 |
| | cylinder for multiple surface aerosol | |
| | generation and method of | |
| | generation | |
| 6,530,370 | Nebulizer apparatus | Mar. 11, 2003 |
| 5,718,222 | Disposable package for use in | Feb. 17, 1998 |
| | aerosolized delivery of drugs | |
| 6,070,575 | Aerosol-forming porous membrane | Jun. 6, 2000 |
| | with certain pore structure | |
| 6,230,706 | Method and device for creating | May 15, 2001 |
| | aerosol with porous membrane with | |
| | certain pore structure | |
| 5,497,763 | Disposable package for | Mar. 12, 1996 |
| | intrapulmonary delivery of | |
| | aerosolized formulations | |
| 5,855,564 | Aerosol extrusion mechanism | Jan. 5, 1999 |
| 7,013,894 | Portable, handheld, pneumatic | Mar. 21, 2006 |
| | driven medicinal nebulizer | |
| 5,379,760 | Position insensitive low resistance | Jan. 10, 1995 |
| | aspirator | |
| 6,899,322 | Method and apparatus for | Dec. 31, 2001 |
| | production of droplets | |
| 7,562,656 | Aerosol medication inhalation | Sep. 17, 2004 |
| | system | |
| 3,762,409 | Nebulizer | Nov. 3, 1970 |
| D471626 | Nebulizer | : Dec. 10, 2001 |
| RE30046 | Nebulizer | Jul. 17, 1979 |
| 5,823,179 | Nebulizer Apparatus and method | Oct. 20, 1998 |
| 5,570,682 | Passive aspiratory nebulizer system | Nov. 5, 1996 |
| 5,685,291 | Nebulizer adapter system for | Nov. 11, 1997 |
| | premature babies | |

Existing nebulizers:

Pneumatic nebulizers have orifices of 500 micrometer and more and other means of separation (extraction) of large droplets, therefore the obtained aerosol is of low concentration, (Concentration=Quantity of droplets per volume), usually less than 10̂7/cm̂3). Such low concentration increases the treatment time in order to achieve use of required dose. In addition there is a hardship to control the dose achieved by existing nebulizers, even some of the most advanced nebulizers entail a synchronization system between the aerosol supply and the respiration rhythm yet there is still aerosol loss and dose accuracy can hardly be maintained.

In the existing nebulizers a significant quantity of atomized liquid is lost in the process of preparing the aerosol due to “dead” volume that can never be sprayed as aerosol.

An additional disadvantage of the existing nebulizers is a mandatory use position (e.g. vertical) to allow proper device operation and to control the amount of the dose.

Another factor to be considered is the exposure of environment to the aerosol (i.e. medicine) while using the existing nebulizers. While patient uses a mask, tent or any other kind of disperser, there is an existing risk of medicine leakage that might be hazardous to environment.

To date the existing nebulizers of ultrasonic type do not allow spraying all kind of liquids since during their process the medicine absorbs ultrasonic energy and heats, which harms the medicine and can distort it. Such nebulizers require high rate supply of external air for liquid cooling and streaming thus creating a relatively low concentrated aerosol (even though higher than the pneumatic type nebulizers) but no more than 10̂8/cm̂3, in best conditions, therefore, the treatment time required, is still high.

The proposed technology resolves the above mentioned problems and overcomes the above encountered disadvantages while offering additional advantages.

### SUMMARY OF THE INVENTION

The proposed technology is intended for creating ultra fine aerosol by using rigid porous material (1) (FIG. 1).

The ultra fine aerosol (0.3 to 1.1 micrometer of droplet size of the aerosol) is achieved due to specific parameters of the porous material, while the porous material acts as a pneumatic multi-nozzle atomizing system. The porous medium itself is in fact an integral system consisting of the following elements:
The liquid (3) for spraying (been on the surface of the porous medium and/or partially absorbed in it)
A large number of pores (2) (of sub-micron size) acting as nozzles.
Gas (4) caged in those pores which are vacant of liquid.

The aerosol spraying is performed by instantaneously inducing pressure drop to the thickness of the porous medium (for instance: when the medium is ‘coin’ shaped the differential pressure occurs between the two flat sides of the ‘coin’) . For example, when the porous medium (which contains the above mentioned liquid and gas) is in atmospheric pressure a sharp pressure drop is induced on one side of the medium (e.g. 600 to 900 mbar less than atmospheric pressure, depending on the porous medium characteristics) (FIG. 2b). In this stage atomizing occurs as a result of the differential pressure (between the two sides of the medium) which causes the medium to act as a pneumatic multi-nozzle. The reason for the effect of pneumatic multi-nozzle atomization is that the air (4) (FIG. 1) which was caged in the internal volume of the porous medium releases itself in the direction of vacuum, causing the pores (2) (FIG. 1) to act as nozzles and spray the liquid (3) (FIG. 1) outside from the medium. The aerosol is then achieved on the side of the lower pressure (atomized side (102a)) (FIG. 2a) with no need for external (atomizing) gas supply.

The under-pressure mentioned above is produced in a chamber, whereas the porous medium is a part of the chamber.

According to the above technology, it is possible to achieve fine aerosol with high concentration (10̂9-10̂11 /crnA3) and more.

The same chamber in which the under-pressure (vacuum) is produced is also used for storing the aerosol, hereby referred to as “the vacuum accumulator”, until the aerosol is required for use (i.e. inhalation), whereas the stability of the aerosol in the chamber depends on its concentration.

In order to take out aerosol (103) (FIG. 2c) from the vacuum accumulator, the pressure in the vacuum accumulator needs to be changed. For example, for an inhalator, an atmospheric pressure has to be produced in such a way that will allow the inhalation of the stored aerosol as a whole with no losses, dosed and repeatable, without need for any synchronization with the respiratory tract.

The vacuum accumulator element may also be used as a drying chamber depending on the relation between the volume of the accumulator chamber and the quantity of the droplets and on the overall parameters of the environment in vacuum accumulator (e.g. temperature, pressure, etc.). In such case dry aerosol can be obtained by the nebulizer (e.g. for purpose of dry particles inhalation, which is a new kind of dry-particle-inhaler that acts without pressurized gas).

Additional drying process can take place by the act of inhalation itself, when the inhaled air acts as drying agent.

The described nebulizer has the following advantages:
The nebulizer can be operated in any environment and any position: upright, horizontal and even in outer space.
Possible of being self sustained.

The porous element may also be used as a storage container for the liquid (i.e. medicine) prior to its conversion to aerosol (can be referred to as “pill”), when it is soaked with a determined quantity of liquid or alternatively when it is normally dry but covered with a buffering (dry) layer (that is not transferable for liquid) packed together with a liquid container (that together with the porous medium form a “sandwich” like device) that has a mechanism of wetting the medium (e.g. by removing the buffering layer) which is performed before the atomizing effect (before the insertion of the “pill” into the nebulizer, or afterwards by an internal mechanism of the nebulizer).

When carried around by user (i.e. prior to atomization process), the “pill” is hermetically and sterilely sealed and packed.

The “pill” is designed to be used with specific nebulizer device. Matching or un-matching combinations can be created to allow or deny certain usage combinations.

Serving as a liquid container the “pill” can contain any desired liquid formulation (e.g. medicine, food supplement, natural sources, etc.) while the “pill” serves as a generic platform for carrying the liquid, ready to be converted into aerosol anytime when fed into a nebulizer device (i.e. for inhalation purposes).

The “pill” can be disposable. It is designed for easy replacement and discharge.

### DESCRIPTION OF THE DRAWINGS

FIG. 1 describes: (1)—porosive material; (2)—nozzles (pores); (3)—liquid for spraying, (4)—gas

FIGS. 2a; 2b; 2c & 3 describe the nebulizer, in different stages: (2a)—when the nebulizer is in idle position, before aerosol is produced; (2b)—the nebulizer is in under-pressure production and aerosol production; (2c)—nebulizer during inhalation act.

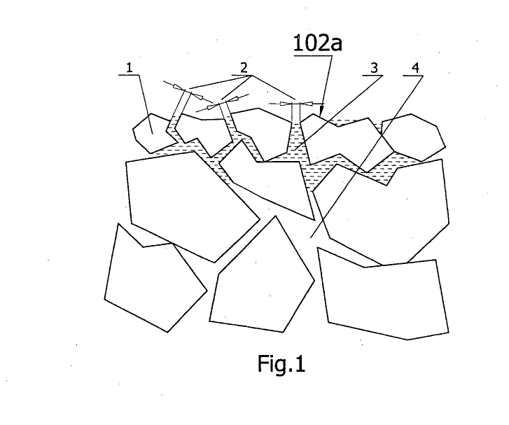

### DETAILED DESCRIPTION

Outer structure (101) containing the vacuum accumulator (101a). The vacuum accumulator's dimensions are determined according to the volume of aerosol required for each application. For example: to produce 30 mg of medical aerosol for local delivery to the lung or systematic delivery through the lung, a volume of 30 cc is required.
The vacuum accumulator has a path (101b) through which the aerosol received from the porous material (102) enters the vacuum accumulator (101a).
The exit hole (103) for aerosol inhalation is externally closed by rotating cover.
Under-pressure effect can be generated either externally by a vacuum pump (for stationary use) or by an internal device of the nebulizer itself, for example, with the help of the cylinder pistol (104) that is moved by spring (105).
On the upper side of the device there is a place to insert the porous medium (102), which may be in the shape of a cylinder, disc, cup or the like, that if fixed or sealed to the device by cover (106), designed for fast opening/closing and has a duct (106b) connecting to environment or vacuum accumulator(101a).

The location of the porous media in relation to the device can be on its upper side (as appears in the drawing) but can also be in the opposite (bottom side) or in any side of the device.

The material, structure and dimensions of the porous material (102) are determined according to the required aerosol characteristics for each required application. For example: the dispersing area determines the quantity of achieved aerosol.

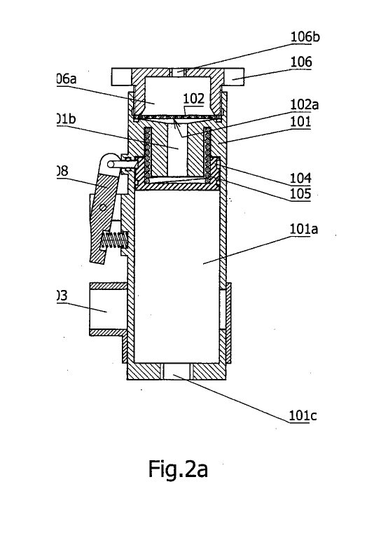

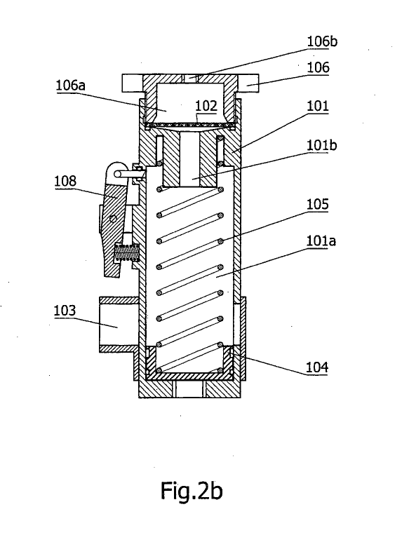

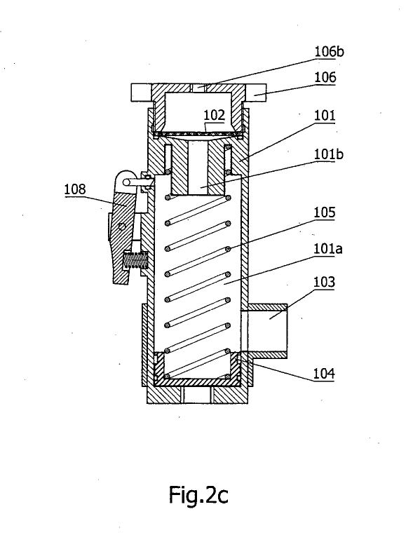

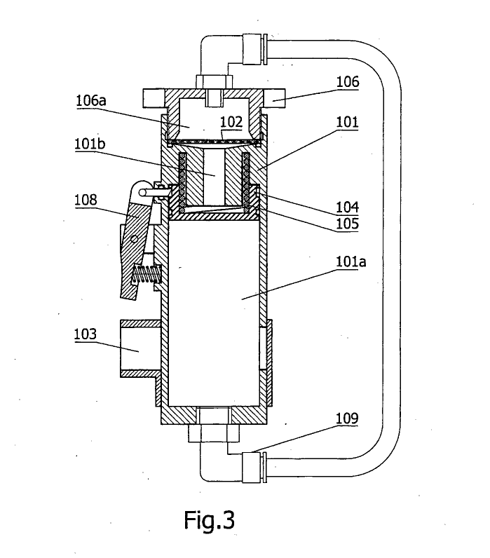

FIG. 2a shows the nebulizer prepared for generating aerosol:
spring (105) is compressed;
piston (104) is captured in upper position with fixture (108);
the porous medium (102) inserts to a place and fixed by cover (106);
the exit hole for aerosol closed by rotating cover (103).

FIG. 2b shows the spraying nebulizer:
piston (104) is—in lower position;
vacuum accumulator (101a) is filled by aerosol.

FIG. 2c shows the nebulizer prepared for aerosol exploitation: rotating cover (103) connecting to environment.

FIG. 3 shows the nebulizer with tube (109) for supply air in spraying stage from vacuum accumulator (101a) to a duct (106b) to increase aerosol capacity by increasing dispersing air. Introducing additional dispersing air causes increase of the differential pressure.

1-10. (canceled)

11. A nebulizer for producing aerosol, comprising a porous medium, wherein the porous medium has two sides and further comprises:
i. a plurality of pores;
ii. a liquid partially adsorbed in the porous medium; and
iii. gas, wherein the gas is caged in pores that are vacant of said liquid, wherein the porous medium is configured to act as a pneumatic multi-nozzle atomizing system.

12. The nebulizer of claim 11, further comprising a chamber and a path, wherein the path is configured to receive the aerosol from the porous medium and transfer said aerosol to the chamber.

13. The nebulizer of claim 11, further comprising an outlet configured to release said aerosol by inhalation.

14. The nebulizer of claim 11, further comprising a cover, configured to open-close the nebulizer.

15. The nebulizer of claim 14, wherein the cover further comprises a duct.

16. The nebulizer of claim 11, further comprising an element configured for creating a pressure drop between the two sides of the porous medium, the element is selected from a piston and spring, and a vacuum pump.

17. The nebulizer of claim 11, wherein the porous medium is in a shape selected from the group consisting of: a cylinder, a disc and a cup.

18. The nebulizer of claim 15, further comprising a tube connecting the chamber to said duct and configured to increase the pressure.

19. The nebulizer of claim 13, wherein said liquid comprises a medication.

20. The nebulizer of claim 12, wherein the chamber is a drying chamber and the aerosol is a dry aerosol.

21. A method of delivering a medication to a subject by inhalation, the method comprising administering the medication to the subject using the nebulizer of claim 11.

22. A porous medium configured to act as a pneumatic multi-nozzle atomizing system, wherein the porous medium has two sides and further comprises:
i. a plurality of pores;
ii. a liquid partially adsorbed in the porous medium; and
iii. gas, wherein the gas is caged in pores that are vacant of said liquid.

23. The porous medium of claim 22, wherein the porous medium is in a shape selected from the group consisting of: a cylinder, a disc and a cup.

24. The porous medium of claim 22, wherein said liquid comprises a medication.

25. A pack, comprising:
(a) a porous membrane having two sides, and further comprises:
i. a plurality of pores; and
ii. a buffering layer, wherein said buffering layer covers said porous membrane, and
(b) a liquid container comprising a medication in a liquid, wherein upon removal of the buffering layer, the liquid is wetting the porous medium.

26. A method for producing ultra fine highly concentrated aerosol, comprising
a. providing the porous medium of claim 11, and
b. inducing a pressure drop between the two sides of the porous medium, thereby producing aerosol, wherein the aerosol is produced in the absence of an external gas supply.

27. The method of claim 26, wherein inducing the pressure drop is achieved by inducing a pressure drop on one side of said two sides of the porous medium.

28. The method of claim 26, wherein inducing the pressure drop is achieved by a vacuum generating mean selected from a vacuum pump and a piston.

29. The method of claim 27, wherein pressure of the porous medium at step (a) is an atmospheric pressure and wherein said pressure drop reduces the atmospheric pressure in said one side by 600 to 900 mbar.

30. The method of claim 26, further comprising storing the aerosol under vacuum.

31. The method of claim 30, wherein the aerosol is transferred and stored under vacuum in a chamber.

32. The method of claim 26, wherein the aerosol concentration is within the range of 10⁹ to 10¹¹ per cm³.

33. The method of claim 26, wherein the liquid comprises a medication.
